(19) United States
(12) Patent Application Publication
(10) Pub. No.: US 2022/0280732 A1
(43) Pub. Date: Sep. 8, 2022

(54) AEROSOL DELIVERY DEVICE WITH AEROSOL SENSOR ASSEMBLY FOR DETECTING THE PHYSICAL AND CHEMICAL PROPERTIES OF THE GENERATED AEROSOL

(71) Applicant: Hao Jiang, Windsor (CA)

(72) Inventor: Hao Jiang, Windsor (CA)

(21) Appl. No.: 17/685,572

(22) Filed: Mar. 3, 2022

Related U.S. Application Data
(60) Provisional application No. 63/155,736, filed on Mar. 3, 2021.

(51) Int. Cl. A61M 15/00; A61M 15/06

(52) U.S. Cl. CPC A61M 15/008; A61M 15/06; A61M 15/0085; A61M 15/009; A61M 2205/3368; A61M 2205/3306; A61M 2205/583; A61M 2205/18; A61M 2205/3553

(57) ABSTRACT

An electronic aerosol safety system including at least one aerosol safety sensor for measuring or sensing parameters of a target in an aerosol stream and a warning device in communication with the sensor, wherein the warning device can provide a user with safety and efficacy information. There is also provided a method of increasing the safety of an aerosol delivery device by attaching or integrating the electronic aerosol safety system into an aerosol delivery system, monitoring the aerosol produced by the aerosol delivery device by detecting preset parameters using the electronic aerosol safety system and triggering a notification to a user of the aerosol delivery device, wherein the triggering occurs upon detection of a deviation from the preset parameters.

### CROSS-REFERENCE TO RELATED APPLICATIONS

This application claims the benefit of U.S. Provisional Application No. 63/155,736, filed on Mar. 3, 2021. The entire disclosure of the above application is incorporated herein by reference.

### FIELD

The present technology relates to an aerosol generating system. More specifically, the present technology relates to an aerosol generating system including an electronic cigarette and an inhalation drug delivery device.

### INTRODUCTION

This section provides background information related to the present disclosure which is not necessarily prior art.

An electronic smoking device, such as an electronic cigarette (e-cig or e-cigarette), electronic cigar, personal vaporizer (PV) or electronic nicotine delivery system (ENDS) is a battery-powered vaporizer which creates an aerosol or vapor. In general, these devices have a heating element that atomizes a liquid solution known as e-liquid. For example, the term e-liquid can be used with regard to vape juice, e-juice, and other tobacco flavored liquids. The main ingredients of e-liquids are usually a mix of propylene glycol (PG), glycerin (G), and/or polyethylene glycol 400 (PEG400), sometimes with differing levels of alcohol mixed with concentrated or extracted flavorings. Optionally, nicotine can be included E-liquid is often sold in bottles or pre-filled disposable cartridges. Pre-made e-liquids are manufactured with various tobacco, fruit, and other flavors, as well as with different concentrations of nicotine.

In some electronic smoking devices, e-liquid is heated at an atomizer to produce an aerosol when the device senses a puff action of a user. The aerosol from an electronic cigarette is generated when a power supply heats a coil housed in an atomizer, which contains a wicking material saturated with the e-liquid formulation. When the coil heats up, the e-liquid in contact with the coil vaporizes, quickly condenses into an aerosol of fine particles, which is then delivered to the user. The aerosol typically is entrained in air flow through a passageway in the device to a mouthpiece or outlet. It is desirable to monitor the amount of the aerosol generated in real-time for the purposes of, for example, controlling the amount of aerosol generated during each puff, and estimating the remaining amount of the e-liquid in the e-liquid cartridge or e-liquid container.

There is currently a great deal of concern about the health risks associated with usage of electronic cigarettes. Electronic cigarettes can be misused in such a manner as to create potential health risks. For example, some devices allow for overheating of the e-liquids to increase nicotine output from the e-liquid. This can create harmful carcinogenic substances including volatile organic compounds and aldehydes, which are generated out of thermal decompositions of e-liquids at too high of a temperature, which can subsequently be inhaled by the user.

A study by Mulder et al. (Scientific Reports, vol. 9, p. 10221, 2019) has demonstrated the strong link between the aerosol particle size distribution and the operating condition of an electronic cigarette, particularly when the generated aerosol contains harmful substances above a predetermined safety limit. If the aerosol particle size distribution, particle concentrations, the aerosol temperature, and/or the composition of a generated aerosol can be measured from inside an electronic cigarette, the potential health risks can be immediately evaluated before the aerosol is inhaled by the user and a warning can be triggered to prevent the user from inhaling the harmful substances.

Inhalation drug delivery devices are used for inhalation therapy to administer medicine through the pulmonary route. A successful inhalation therapy requires a harmonic interaction between the drug formulation, the inhalation drug delivery device, and the patient (Ibrahim et al., Med Devices, vol. 8, pp. 131-139, 2015). However, the incorrect use of the device due to lack of training in how to use the device or how to coordinate actuation and aerosol inhalation has often compromised the efficacy of the inhalation therapy. If aerosol sensors can be installed into the inhalation drug delivery devices to monitor the physical and chemical characteristics of the generated aerosol, the collected information can be used to assist and guide the user to use the device properly. Popular inhalation drug delivery devices include, but are not limited to, medical nebulizer, pressurized metered-dose inhaler, and dry powder inhaler.

Accordingly, there is a need to develop a system for internally monitoring aerosol particle size distribution and the operating condition of an electronic cigarette and an inhalation drug delivery device.

### SUMMARY

In concordance with the instant disclosure, an electronic aerosol safety system, has surprisingly been discovered.

The present technology includes articles of manufacture, systems, and processes that relate to an electronic aerosol safety system for an aerosol delivery system. The electronic aerosol safety system can include an aerosol sensor assembly which can include at least one of the following: an aerosol particle sensor, a chemical sensor, and a temperature sensor. The aerosol sensor assembly can be installed onto the aerosol delivery channel to measure the content of the generated aerosol before being inhaled by a user. The measured parameters of the generated aerosol can include aerosol particle size distribution, particle concentrations, chemical compositions, and/or aerosol temperature. The measured parameters of the generated aerosol can be displayed on an information display and stored within the system. The data of the measured parameters can be sent to a connected mobile device through a wireless communication module.

The electronic aerosol safety system can be consolidated inside the housing of an aerosol delivery system. Alternatively, the electronic aerosol safety system can be formed as a detachable adapter for connection to an aerosol delivery system.

There is provided a method of operating the electronic aerosol safety system of the present disclosure to detect the presence of harmful substances. If harmful substances measured from the generated aerosol exceed a preset threshold, a warning message can be immediately displayed on an information display. In some embodiments, a vibration motor can also be triggered to vibrate. Further, a push notification with a warning message can also be triggered on a connected mobile device, to warn the user of potential health risks.

The electronic aerosol safety system can also provide a method of operating an aerosol delivery device of the present disclosure to detect and/or control the quality of the generated aerosol. Desired parameters for the aerosol can be determined in accordance with the type of e-liquid, the type of atomizer, manufacturer's recommendations, user's selection, preferences, and/or habits. The measured parameters from the generated aerosol can be compared with the desired parameters, and new settings for the atomizer controller module can be calculated and applied for generating an aerosol with parameters matching with the desired parameters. The settings for the atomizer controller module can be updated every time when a user inhales the aerosol.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic view of an aerosol delivery device according to an embodiment of the present disclosure;

FIG. 2 is a schematic view of an aerosol delivery device according to an embodiment of the present disclosure;

FIG. 3 is a schematic view of an aerosol particle sensor according to an embodiment of the present disclosure;

FIG. 4 is a schematic view of a medical nebulizer containing an aerosol particle sensor according to an embodiment of the present disclosure;

FIG. 5A is a schematic view of an aerosol particle sensor according to an embodiment of the present disclosure;

FIG. 5B is a schematic view of the aerosol particle sensor of FIG. 5A;

FIG. 6 is a schematic view of an aerosol particle sensor for use in the device according to an embodiment of the present disclosure;

FIG. 7A is a schematic view of an aerosol particle sensor having an optical module that includes a light source as one or more LEDs and a photodetector according to an embodiment of the present disclosure;

FIG. 7B is a schematic view of the optical module of the aerosol particle sensor of FIG. 7A;

FIG. 8 is a schematic view of an aerosol particle sensor with an aerosol diluter for use in the aerosol delivery device according to an embodiment of the present disclosure;

FIG. 9 is a schematic view of a temperature sensor for use in the aerosol delivery device according to an embodiment of the present disclosure;

FIG. 10 is a schematic view of a chemical sensor for use in the aerosol delivery device according to an embodiment of the present disclosure;

FIG. 11 is a schematic view of an alternative chemical sensor for use in the aerosol delivery device according to an embodiment of the present disclosure;

FIG. 12 is a schematic view of a mobile device wirelessly connected with the aerosol delivery device of the present disclosure;

FIG. 13 is a flow chart showing a method of operating an aerosol delivery device according to an embodiment of the present disclosure;

FIG. 14 is a flow chart showing a method of operating an aerosol delivery device according to an embodiment of the present disclosure;

FIGS. 15A and 15B are graphs depicting the results of using an electronic aerosol safety system according to an embodiment of the present disclosure; and

FIG. 16 is a graph depicting the results of using an electronic aerosol safety system according to an embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below,” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves the safety and efficacy of aerosol generating devices. In particular, the present technology improves the safety and efficacy of aerosol generating devices by incorporating an aerosol sensor to monitor the physical and chemical characteristics of the generated aerosol. Specifically, the present technology improves electronic cigarettes by measuring aerosol particle size distribution and particle concentrations, aerosol temperature, and harmful chemicals. The present technology also improves inhalation drug delivery devices by measuring aerosol particle size distribution and particle concentrations, aerosol temperature, and concentration of chemicals.

Provided are embodiments of an aerosol safety system that can include various features and that can be used in various ways. The safety system of the present disclosure can be used for monitoring the quality of the generated aerosol for promoting user experiences in vaping. The safety system can be used for protecting users from vaping too harmful chemicals and/or low-quality aerosols, and for minimizing the health risks in vaping. Additionally, the safety system can be used for tracking the usage history and aiding a user to achieve cessation of vaping. Further, the safety system can be used for studying certain respiratory and circulatory diseases related to vaping. The safety system can also be used to improve the efficacy of the inhalation drug delivery device to which it is attached.

In certain embodiments, the safety system according to the present disclosure can be incorporated into the body of an aerosol generating device or it can be a separate part that is attached or affixed to an existing aerosol generating device. Additionally, the safety system can be used in connection with any material or product that is capable of being aerosolized. Examples of such materials are well known to those of skill in the art. More specifically, the safety system can include an aerosol sensor assembly 50. The aerosol sensor assembly 50 can sense a range of predetermined safety and efficacy parameters. By way of example, these parameters can include, but are not limited to, aerosol particle size distribution, particle concentrations, chemical compositions, and/or aerosol temperature. Additional parameters known to those of skill in the art can also be sensed without departing from the spirit of the present disclosure.

The aerosol sensor assembly 50 can include at least one of the following: an aerosol particle sensor 60 for measuring aerosol particle size distribution and particle concentrations, a temperature sensor 70 for measuring the aerosol temperature, and/or a chemical sensor 80 for detecting chemicals in the generated aerosol. The aerosol sensor assembly can include one or more of the above referenced sensors, wherein the inclusion of specific sensors can vary depending on the specific location of the aerosol sensor assembly 50 as well as the intended use. Such modifications can be made by one of skill in the art.

The aerosol sensor assembly 50 can also include one or more warning or alert functionalities. The warning or alert functionalities can be implemented using a microcontroller board 30 including a microcontroller 32, data storage 34, a wireless communication module 36, a vibration motor 40 for warning a user of potential health risks with vibration, an information display 42 for displaying parameters of the generated aerosol and warning messages, and/or at least one data cable 44 for connecting electronic components.

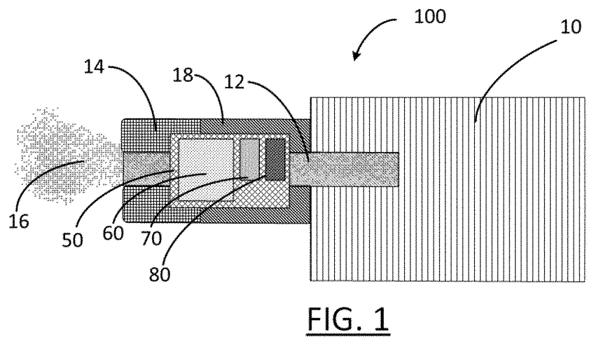

By way of example, FIG. 1 depicts an aerosol delivery device 100 including the safety system of the present disclosure, where all of the components can be self-contained or assembled inside a single housing. The aerosol delivery device 100 can include an aerosol generator 10, an aerosol delivery channel 12, a mouthpiece 14, and an aerosol sensor assembly 50. The aerosol 16 can be generated from the aerosol generator 10, can travel through the aerosol delivery channel 12 and the mouthpiece 14, and can then be inhaled by a user. The aerosol delivery device 100 can further include a connector 18 to connect portions of the individual components. The aerosol sensor assembly 50 can include an aerosol particle sensor 60 for measuring aerosol particle size distribution and particle concentrations, a temperature sensor 70 for measuring the aerosol temperature, and a chemical sensor 80 for detecting chemicals in the generated aerosol prior to inhalation by the user.

The aerosol delivery device 100 can be configured as an electronic cigarette device, wherein the aerosol generator 10 can be an atomizer 22. As used herein, the term “electronic cigarette” can be used to also reference an electronic smoking device, an electronic cigar, a personal vaporizer, a vaping device, a vape mod, an electronic hookah, and an electronic nicotine delivery system. Alternatively, the aerosol delivery device 100 can be configured as an inhalation drug delivery device 400. Examples of such devices include, but are not limited to, a medical nebulizer, a pressurized metered-dose inhaler, and a dry powder inhaler.

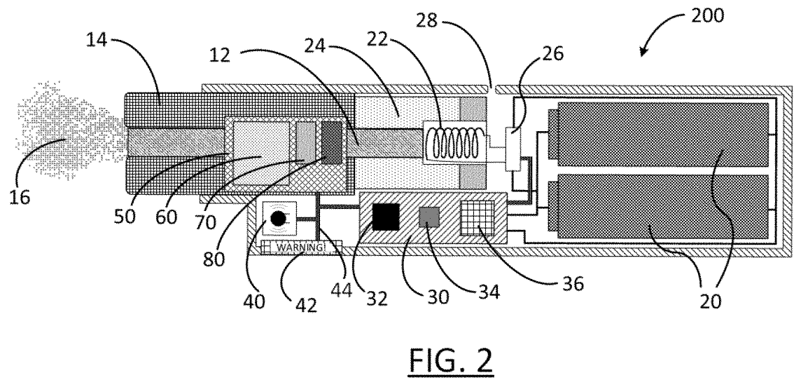

FIG. 2 shows an embodiment wherein the aerosol delivery device 100 is an electronic cigarette device 200 including the safety system of the present disclosure, with all components assembled inside a single housing. The electronic cigarette device 200 can include a power supply 20 comprising batteries for powering up the device, an atomizer 22 for atomizing an e-liquid into an aerosol, a reservoir 24 for storing the e-liquid, an atomizer controller module 26 for controlling the operating condition of the atomizer 22. Further provided are an air inlet 28, an aerosol delivery channel 12 for delivering the generated aerosol 16 from the atomizer 22 into a user's mouth, and a mouthpiece 14. A microcontroller board 30 is included that has a microcontroller 32, data storage 34, a wireless communication module 36. A vibration motor 40 is provided for warning a user of potential health risks with vibration along with an information display 42 for displaying one or more parameters of the generated aerosol and warning messages. At least one data cable 44 is included for connecting the electronic components. An aerosol sensor assembly 50 is included that has an aerosol particle sensor 60 for measuring aerosol particle size distribution and particle concentrations, a temperature sensor 70 for measuring the aerosol temperature, and a chemical sensor 80 for detecting chemicals in the generated aerosol.

More specifically, the aerosol delivery channel 12 can include the opening in the reservoir 24, the opening in the mouthpiece 14, and the opening in any component between the reservoir 24 and the mouthpiece 14. The aerosol sensor assembly 50 can be located within the aerosol delivery channel 12 for measuring particulate matter within the generated aerosol before inhalation by the user. While the aerosol sensor assembly 50 is positioned within the aerosol delivery channel 12, the aerosol sensor assembly 50 neither blocks nor impacts the flow of the generated aerosol within the aerosol delivery channel 12.

As used herein, the term “atomizer” can be used to also reference a cartomizer and/or clearomizer without departing from the spirit of the present disclosure. The atomizer 22 can heat the e-liquid to a temperature required for atomization. However, other technologies and systems for atomization can also be used. One non-limiting example of such a system is an ultrasonic atomizer, which can use ultrasonic high-frequency resonance. Alternatively, the atomizer 22 and the reservoir 24 can be combined into a single component. Additionally, a variety of compositions and compounds can also be used with the atomizers. These can include any composition or compound known to those of skill in the art capable of being inhaled using an atomizer, cartomizer, and/or clearomizer.

The aerosol delivery device 100 as described herein can also include additional components without departing from the spirit of the disclosure. Examples of such components can include, but are not limited to, a push button, USB connector, battery charging port, light emitting diode (LED), activation switch, cover, handle, and/or case.

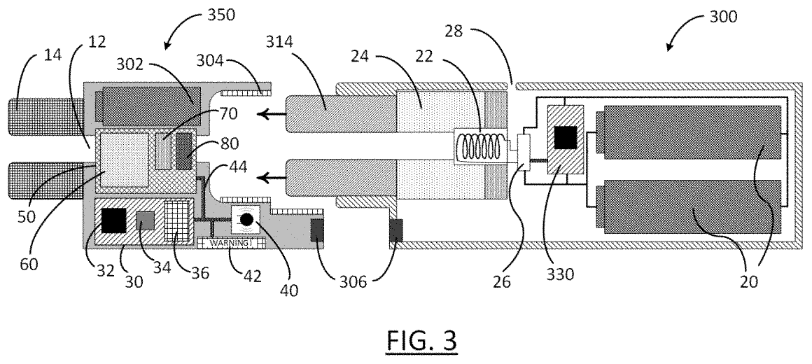

In an alternative embodiment, the aerosol delivery device 100 can include the safety system of the present disclosure as a separate, attachable component. As shown in FIG. 3, an electronic cigarette device 200 can include an aerosol generator 300 and a detachable adapter 350. The aerosol generator 300 can include the power supply 20, the atomizer 22, the reservoir 24, the atomizer controller module 26, the air inlet 28, an aerosol outlet 314 on the aerosol generator 300, a microcontroller board 330 for controlling the operation of the aerosol generator 300, and a connector fixture 306. The detachable adapter 350 can include the mouthpiece 14, the microcontroller board 30 including the microcontroller 32, the data storage 34, and the wireless communication module 36, the vibration motor 40, the information display 42, the data cable 44, the aerosol sensor assembly 50 including the aerosol particle sensor 60, the temperature sensor 70, and the chemical sensor 80, a secondary battery 302 for powering up the detachable adapter 350, a connector 304 with air-tight seal, and a connector fixture 306.

More specifically, the detachable adapter 350 can be mounted on the aerosol generator 300 by inserting the aerosol outlet 314 into the connector 304, wherein the air-tight seal on the inner wall of the connector 304 is pressed onto the aerosol outlet 314 thereby forming an air-tight connection. After mounting the detachable adapter 350 onto the aerosol generator 300, the connector fixture 306 on the detachable adapter 350 can be attached to the connector fixture 306 on the aerosol generator 300 thereby securing the connection. The connector fixture 306 can include a paired piece (not shown) on the aerosol generator 300 for engaging the connector fixture 306 on the detachable adapter 350. The pairing can be based on a mechanical or magnetic relationship between the pieces. After mounting the detachable adapter 350 onto the aerosol generator 300, an aerosol delivery channel 12 can be formed by the connection of the openings of the detachable adapter 350, the mouthpiece 14, the aerosol outlet 314, and the reservoir 24. The aerosol sensor assembly 50 can be attached onto the aerosol delivery channel 12 for measuring the generated aerosol prior to inhalation by the user. The aerosol sensor assembly 50 does not block the flow of the generated aerosol in the aerosol delivery channel 12.

Certain variations can be made to the above-described electronic cigarette device without deviating from the spirit of the present disclosure. The aerosol generator 300 can be a regular commercial electronic cigarette and the aerosol outlet 314 can be configured as a mouthpiece for coupling to the regular commercial electronic cigarette. The detachable adapter 350 can be customized to fit with different brands and models of regular electronic cigarettes. The detachable adapter 350 can also be powered by the power supply 20 of the aerosol generator 300 via additional electrical connections between the aerosol generator 300 and the detachable adapter 350.

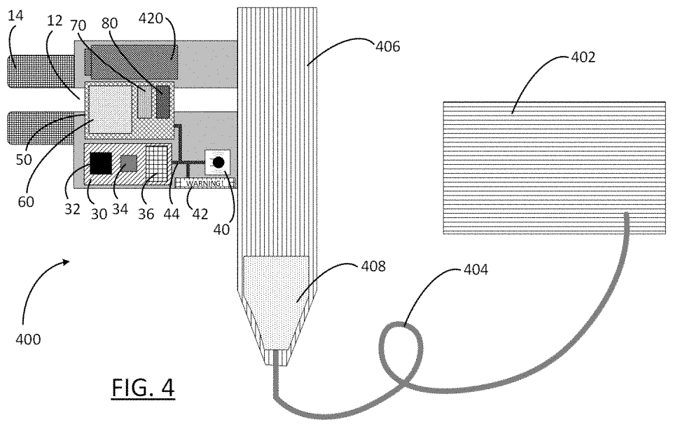

In another embodiment, as shown in FIG. 4, the aerosol delivery device 100 can be a medical nebulizer 400. In this embodiment, the aerosol sensor assembly 50 can enable the medical nebulizer 400 to function in a more efficacious manner. The aerosol sensor assembly 50 can provide efficacy information instead of merely safety information. The medical nebulizer 400 can include a compressor 402, tubing 404, a nebulizer cup 406, the aerosol delivery channel 12, the mouthpiece 14, the microcontroller board 30 comprising the microcontroller 32, the data storage 34, and the wireless communication module 36, a battery 420, the vibration motor 40 for warning a user of low-quality aerosols, the information display 42 for displaying parameters of the generated aerosol and warning messages, the data cable 44 for connecting electronic components, and the aerosol sensor assembly 50 comprising the aerosol particle sensor 60 for measuring aerosol particle size distribution and particle concentrations, the temperature sensor 70 for measuring the aerosol temperature, and the chemical sensor 80 for detecting chemicals in the generated aerosol. A solution 408 including one or more active ingredients (e.g., pharmaceuticals) can be loaded inside the nebulizer cup 406 and can become aerosolized by the pressurized flow of air from the compressor 402. The generated aerosol can be delivered to the user through the aerosol delivery channel 12. Variations can be made to the medical nebulizer 400, without departing from the spirit of the present disclosure. For example, the compressor 402, the tubing 404, and the nebulizer cup can be combined into a single compact handheld component. Additional components can also be added. Some non-limiting examples include, but are not limited to, push buttons, a USB connector, a battery charging port, LEDs, an activation switch, a cover, a handle, and/or a case.

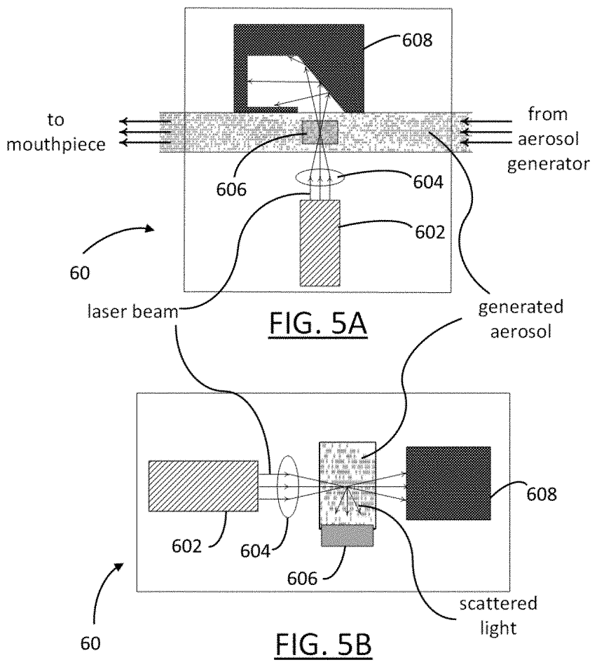

Specific reference is now made to FIG. 5A and FIG. 5B, which show an optical configuration of an aerosol particle sensor 60. FIG. 5A is the top view schematic of the configuration and FIG. 5B is the side view of the same configuration. The aerosol particle sensor 60 can include a laser diode 602, a focal lens 604, a photodetector 606, and a light trap 608. There can typically be two principles for sensing particle sizes using a laser beam, including optical particle counting (OPC) and dynamic light scattering (DLS). Laser beam output from the laser diode 602 can be focused by the focal lens 604 onto a spot inside the aerosol delivery channel 12 to interact with the generated aerosol inside the channel and the diverging beam after the focus spot can be collected and absorbed by the light trap 608. The photodetector 606 can be positioned underneath the aerosol to collect the laser light scattered by aerosol particles in the generated aerosol and an analog electrical signal is generated which is proportional to the intensity of the scattered light. The analog electrical signal can be amplified, filtered, and then converted into a digital signal using analog-to-digital conversion (ADC).

In the principle of OPC, each particle travelling across the laser beam causes a pulse in the signal. A larger particle can generate a higher pulse while a smaller particle can generate a lower pulse. The entire signal can be processed using pulse height analysis (PHA) to calculate the particle size distribution and particle concentrations in different size channels. In the results of the computation, particle size channels can be composed of 6 channels, including particle size between 0.3 micrometer and 0.5 micrometer, particle size between 0.5 micrometer and 1 micrometer, particle size between 1 micrometer and 2.5 micrometer, particle size between 2.5 micrometer and 5 micrometer, particle size between 5 micrometer and 10 micrometer, and particle size larger than 10 micrometers.

In the principle of DLS, the particle size distribution and particle concentrations can be computed by calculating an auto-correlation function (ACF) first, and then fitting the particle concentrations of multiple particle size channels into the ACF. The algorithms that can be used in data fitting include, without limitation, CONTIN and CUMULANT.

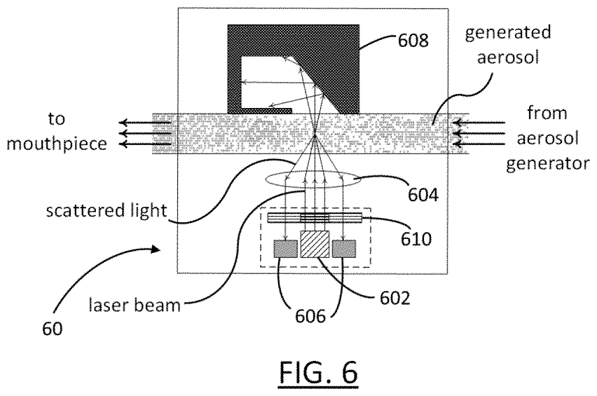

In another embodiment of the aerosol particle sensor, as shown in FIG. 6, the aerosol particle sensor 60 can detect particles based on OPC or DLS. Under this configuration, the laser diode 602 and the photodetector 606 can be positioned closely from the same side of the focal lens 604. The laser beam output from the laser diode 602 can be focused by the focal lens 604 onto a spot inside the aerosol delivery channel 12 to interact with the generated aerosol inside the channel and the diverging beam after the focus spot is collected and absorbed by the light trap 608. The back-scattered light from aerosol particles can be routed by the same focal lens 604 onto the photodetector 606 and an analog electrical signal can be generated which is proportional to the intensity of the scattered light. A flat and transparent layer 610 of a holographic optical element (HOE) or a diffractive lens can be inserted between the laser diode 602 and the focal lens 604, for routing the laser light with high optical efficiencies.

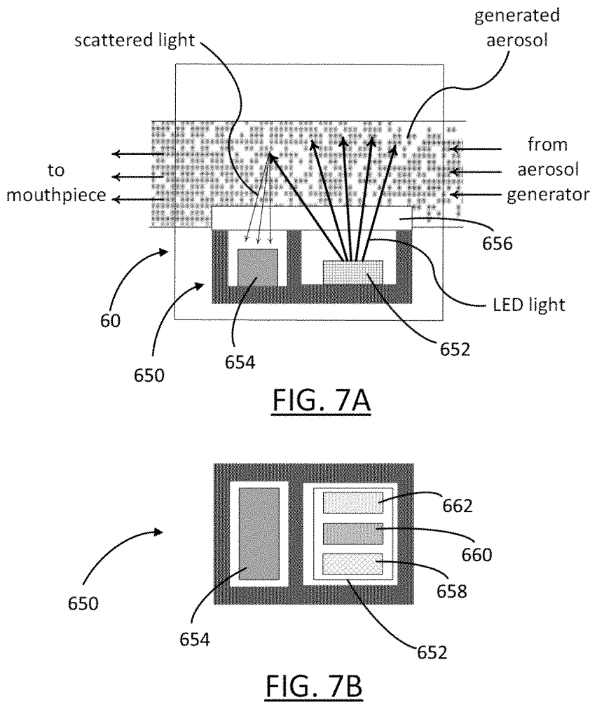

Alternatively, as shown in FIG. 7A and FIG. 7B, the aerosol particle sensor 60 can be capable of detecting particle concentrations of at least two particle size channels based on the principle of photometric measurement. The aerosol particle sensor 60 can include an optical module 650 which can include an LED assembly 652, a photodetector 654, and a transparent cover glass 656. The LED assembly 652 can further include at least three LEDs: LED 658, LED 660, and LED 662. The emission spectra of these three LEDs can be configured to have no overlap. For example, LED 658 can emit light around 880 nm wavelength, LED 660 can emit light around 660 nm wavelength, and LED 662 can emit light around 527 nm wavelength. The three LEDs can be switched on and off periodically and alternatively, and in any given instant, only one LED is turned on. The light output from the LED of “ON” state can reach the aerosol particles inside the aerosol delivery channel 12, and the scattered light can be collected and measured by the photodetector 654.

The mechanism of sensing particle size distribution can be based on contrast in optical scattering efficiencies for light of different wavelengths scattered from aerosol particles of different particle size channels. Provided the intensity measured from LED 658, LED 660, and LED 662 for wavelength 880 nm (λ₁), 660 nm (λ₂), and 527 nm (λ₃), is given by I_{∥1}, I_{λ2}, and I_{λ3}, each measured intensity can be proportional to the mass concentration of the aerosol particles. However, each wavelength can have a specific higher sensitivity on particles of a certain size range. The longer wavelength can primarily measure the larger particles while the shorter wavelength mainly measures smaller particles. For example, the mass concentration of particles larger than 1 micrometer can be estimated by I_{λ1} (for wavelength 880 nm) while the mass concentration of particles smaller than 1 micrometer can be estimated by I_{λ3} (for wavelength 527 nm).

Certain variations can be made to the above-described aerosol particle sensor without departing from the spirit of the present disclosure. The optical module 650 can contain a single LED, and the acquired signal from photodetector 654 can be used to calculate the concentration of aerosol particles for a single particle size channel, which covers all particles with size between 0.05 micrometer and 20 micrometers. The optical module 650 can also contain two LEDs of different wavelengths, and the acquired signal from photodetector 654 can be used to estimate the concentration of aerosol particles of two particle size channels.

According to the study by Floyd et al. (PLOS ONE, vol. 13, No. 12, e10221, 2018), when the atomizer 22 of an electronic cigarette device 200 is overheated, high-concentration harmful chemicals can be produced, and larger particles can take a bigger portion in the generated aerosol. To evaluate the condition for comparing the portion of smaller particles with bigger particles, a particle size distribution index (PSDI) is given as:

$$PSDI = \frac{{a}_{0} {PM}_{x}}{{PM}_{20} - {PM}_{x}} \qquad ( 1 )$$

where PMₓ can stand for mass concentration of particles smaller than x micrometer, PM₂₀ stands for mass concentration of particles smaller than 20 micrometers. For example, when x=1.0, the PSDI can be proportional to the ratio of mass concentration of particles smaller than 1 micrometer to the mass concentration of particles in the size of 1.0 micrometer-20 micrometers. The coefficient α₀ can be a constant to be calibrated for each sensor to normalize the index.

Given the aerosol particle sensor 60 configuration including the laser diode 602 and the photodetector 606 (in FIG. 5 and FIG. 6), the values of PMₓ and PM₁₀ can be directly calculated from the measured particle size distribution in all size channels.

Given the aerosol particle sensor 60 including the LED assembly 652 and the photodetector 654 (in FIG. 7), the PSDI can be approximated by the measured light intensity from two different wavelengths as:

$$PSDI = \frac{{b}_{0} {I}_{λ 3}}{{I}_{λ 1}} \qquad ( 2 )$$

The coefficient b₀ can be a constant to be calibrated for each sensor to normalize the index. A higher PSDI can represent an aerosol with larger portion of smaller particles, which reflects an aerosol of higher quality. A safety threshold for the PSDI can be determined to evaluate the quality of the aerosol. If the measured PSDI is higher than the safety threshold, the aerosol can be considered to be generated with a proper heating power and safe to inhale. Otherwise, the aerosol can be considered to be overheated as it includes too many larger particles and is harmful for the health.

If the aerosol particle sensor includes a single LED at wavelength Lo, the PSDI can be defined to be inversely proportional to the scattered light intensity I_{λ0}, given by:

$$PSDI = \frac{{c}_{0}}{{I}_{λ 0}} \qquad ( 3 )$$

The coefficient c₀ is a constant to be calibrated for each sensor to normalize the index. A too low PSDI represents an aerosol with too high concentration of particles, which often indicates an overheated atomizer and a harmful aerosol.

Certain variations can be made to the above-described calculation of the PSDI, without departing from the spirit of the present disclosure. For example, the PSDI in equation (2) can also be modified into:

$$PSDI = \frac{{m}_{0} {I}_{λ 3}}{{n}_{0} {I}_{λ 1} - {k}_{0} {I}_{λ 3}} \qquad ( 4 )$$

The coefficient m₀, n₀ and k₀ are constants to be calibrated for each sensor to normalize the index. A higher PSDI can represent an aerosol with larger portion of smaller particles, which reflects an aerosol of higher quality.

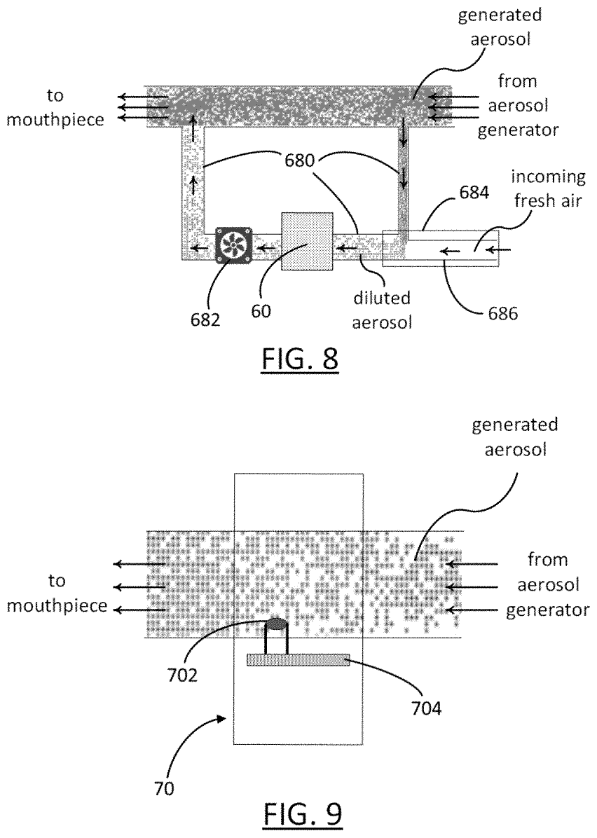

A further embodiment of the aerosol particle sensor 60 is shown in FIG. 8, wherein the aerosol particle sensor 60 can be installed onto a branch 680 of the aerosol delivery channel 12. A portion of the generated aerosol can enter and flow through the branch 680, which can be measured by the aerosol particle sensor 60. A miniature electric axial fan 682 can be installed on the branch 680 to drive the flow of the aerosol in the branch 680. In an aerosol generated by an electronic cigarette, the particle concentrations can often be too high to be accurately detected using above-described optical approaches. In order to improve the accuracy of the aerosol particle sensor 60, an aerosol diluter 684 can include an airflow channel 686 that can be connected to the branch 680. Incoming fresh air can enter the airflow channel 686 and can be mixed with a portion of aerosol in the branch 680 for effectively diluting the aerosol. The diluted aerosol can then be measured by the aerosol particle sensor 60. The airflow channel 686 can be connected to the air inlet 28 or can be connected to a different opening on the device, to enable intake of fresh air from outside the device. The dilution ratio of the aerosol can be controlled by the configuration of the branch 680 and the airflow channel 686.

FIG. 9 shows an embodiment wherein the aerosol sensor assembly 50 can include a temperature sensor 70. The temperature sensor 70 can include a thermistor 702, which can be a variable resistor with resistance value in response to temperature, and an electronic circuit board 704. The thermistor 70 can contact the generated aerosol in the aerosol delivery channel 12 and measure the aerosol temperature. The aerosol temperature can directly reflect the physical condition of the generated aerosol. A very high aerosol temperature can indicate an overheated atomizer, which can produce harmful chemicals. In addition, a very high temperature aerosol can also cause unpleasant pain in the user's mouth and throat. There is a safety threshold determined for the aerosol temperature, and if the measured temperature is above the threshold, the user can be immediately warned about the harmful aerosol.

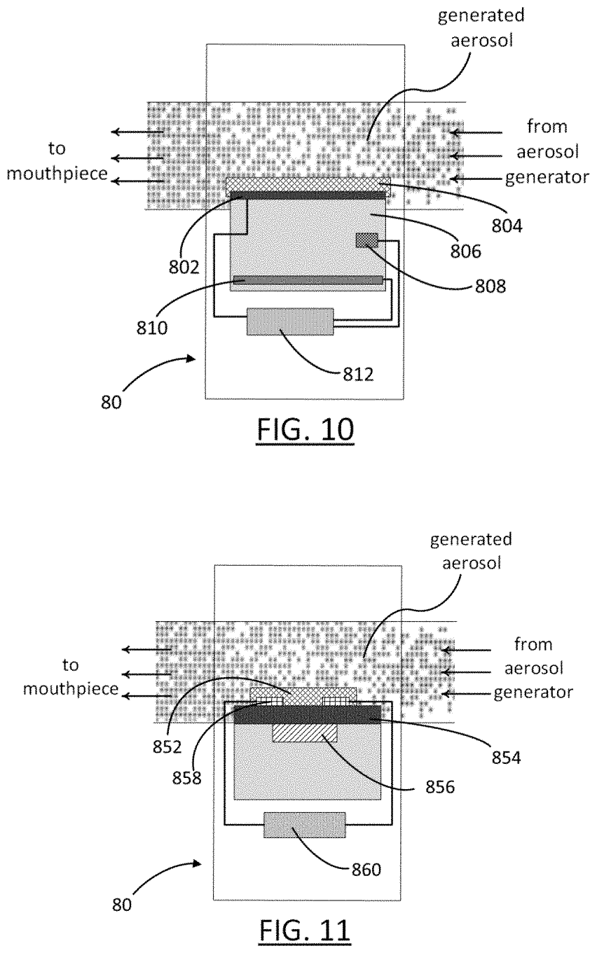

FIG. 10 shows an embodiment wherein the aerosol sensor assembly 50 can include a chemical sensor 80 that can function as an electrochemical cell. The chemical sensor 80 can include a sensing electrode 802 coated with a sensing membrane 804, an electrolyte 806, a reference electrode 808, a counter electrode 810, and an electronic circuit board 812. The sensing membrane 804 can include chemical and physical properties selective to one or more target chemicals. When target chemicals are present in the generated aerosol, they can be captured by the sensing membrane 804 and a potential difference between the sensing electrode 802 and the reference electrode 808 can be induced and detected by the electronic circuit board 812.

FIG. 11 shows an embodiment of the chemical sensor 80 based on a metal oxide (MOX) sensor configuration. The chemical sensor 80 can include a metal oxide sensing layer 852 on top of a substrate 854, a heater 856, electrodes 858, and an electronic circuit board 860. The sensing layer 852 can be heated by the heater 856 that can cause a redox reaction when one or more target chemicals in the aerosol come in contact with the sensing layer 852, changing the electrical resistance across the sensing layer 852. The change in electrical resistance can be measured via the electrodes 858 and the electronic circuit board 860. A higher concentration of chemical can give a lower electrical resistance.

Target chemicals to be measured by the chemical sensor 80 can include, without limitation, nicotine, flavoring agents, and volatile organic compounds (VOCs), in order to monitor the health risks for a user. In order to detect more than one chemicals, multiple sensor elements can be included in the chemical sensor 80, with each sensor element detecting one target chemical.

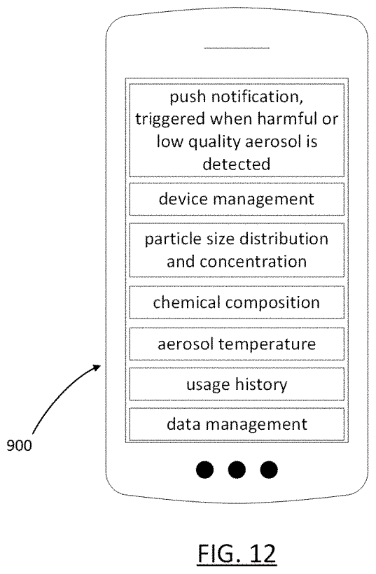

In another embodiment, shown in FIG. 12, a mobile device 900 can be wirelessly connected with the aerosol delivery device 100 of the present disclosure. The mobile device 900 can receive data from the aerosol sensor assembly 50 regarding the measured parameters and can store the data in data storage within the mobile device 900, this can further include time stamps associated with the data. The measured parameters of the aerosol, including particle size distribution and particle concentrations, PSDI, chemical compositions, and aerosol temperature, can be displayed on the screen of the mobile device 900. If the measured concentration of chemicals in the generated aerosol is above a safety threshold, if the PSDI is below a safety threshold, and/or if the aerosol temperature is above a safety threshold, a push notification can be triggered and displayed on the screen accompanied with vibration of the mobile device 900. The software installed on the mobile device 900 can also allow a user to manage the wireless connection with the electronic cigarette device, usage history, and usage data. Further, the software installed on the mobile device 900 can also allow the user to enter the desired parameters of the aerosol which can be used in method 1100. The types of mobile device 900 that can be used with this system can include, but are not limited to, a smartphone, a fitness tracker, a smart watch, a tablet, a laptop, or any other electronic device containing wireless communication modules and microprocessors.

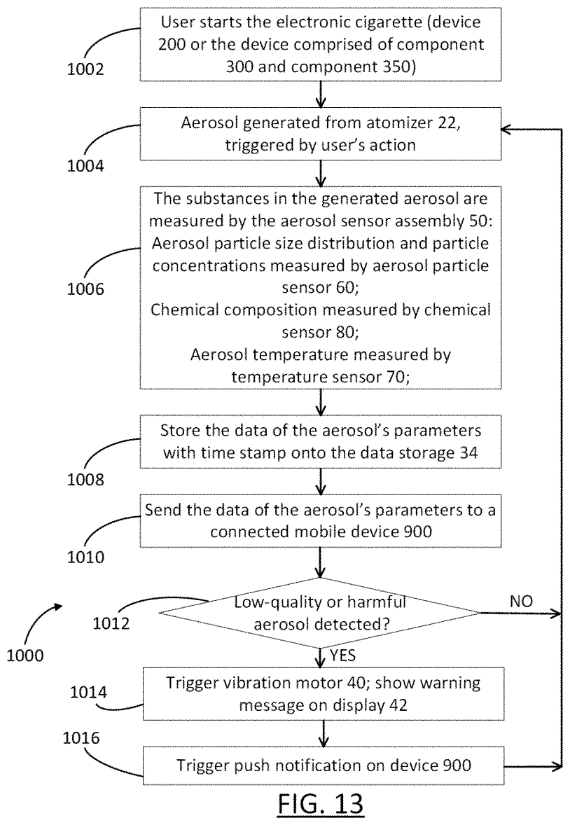

As shown in FIG. 13, a method 1000 is shown for operating the aerosol delivery device 100 including the safety system of the present disclosure, where low-quality or harmful aerosol can be detected in order to minimize health risks for the user. At event 1002, a user can start the aerosol delivery device 100. At event 1004, a user can inhale on the device 10 and the atomizer 22 can be activated to generate the aerosol. At event 1006, the parameters of the generated aerosol can be measured from the aerosol sensor assembly 50. The aerosol particle size distribution and particle concentrations can be measured by the aerosol particle sensor 60. The aerosol temperature can be measured by the temperature sensor 70. The chemical compositions can be measured by the chemical sensor 80. At event 1008, all the measured parameters can be stored on the data storage 34 with time stamps. At event 1010, the data of the measured parameters can be sent to the connected mobile device 900 through the wireless communication module 36. At event 1012, the parameters measured by the aerosol sensor assembly 50 can be analyzed to determine if the aerosol is of low quality or harmful. If the measured concentration of chemicals in the generated aerosol is above the safety threshold, if the PSDI is below the safety threshold, and/or if the aerosol temperature is above the safety threshold, the aerosol can be determined to be of low quality or harmful, and the user can be warned immediately (event 1014 and 1016) in order to prevent the user from inhaling the generated aerosol. At event 1014, the vibration motor 40 can be triggered to vibrate to warn the user to stop vaping, and a warning message can be shown on the information display 42. A push notification can also be triggered on the connected mobile device 900. If the aerosol can be determined to be safe to inhale, the user can continue to inhale the generated aerosols as given at event 1004.

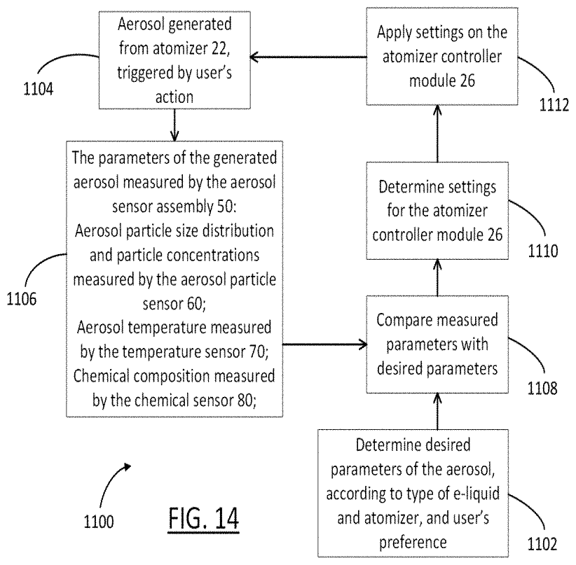

Alternatively, as shown in FIG. 14, a method 1100 of operating the electronic cigarette device including the safety system of the present disclosure can be used for controlling the quality and parameters of the generated aerosol. At event 1102, desired parameters of the aerosol can be configured according to the type of e-liquid, type of atomizer, manufacturer's recommendations, and user's selection, preferences, and/or habits. These desired parameters can also be entered by the user from the software installed on the wirelessly connected mobile device 900. These desired parameters can be predetermined based on experiments on the products or drugs and can be stored within the data storage 34. Each product or drug can be selected from the software installed on the wirelessly connected mobile device 900 to configure the parameters automatically. At event 1104, a user can inhale on the device and the atomizer 22 can be activated to generate the aerosol. At event 1106, the parameters of the generated aerosol can be measured from the aerosol sensor assembly 50. The aerosol particle size distribution and particle concentrations can be measured by aerosol particle sensor 60. The aerosol temperature can be measured by the temperature sensor 70. The chemical compositions can be measured by the chemical sensor 80. At event 1108, the measured parameters can be compared with desired parameters to evaluate the differences between the generated aerosol and the ideal aerosol. At event 1110, an algorithm can be implemented to calculate one or more new settings for the atomizer controller module 26, including wattage, duration, and waveform to be applied on the atomizer 22. At event 1112, the one or more new settings can be applied onto the atomizer controller module 26, to improve the quality of the generated aerosol to reach the desired characteristics. Every time when a user inhales the aerosol, settings can be updated according to the closed loop of events 1104, 1106, 1108, 1110, 1112, and 1104.

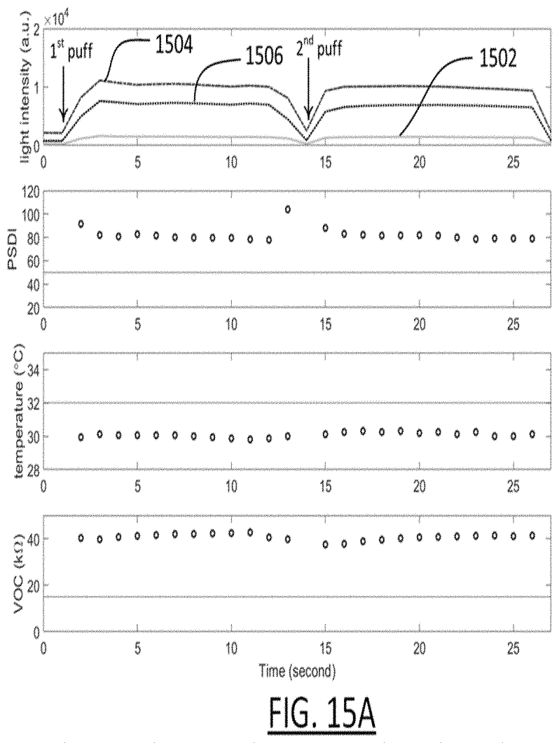

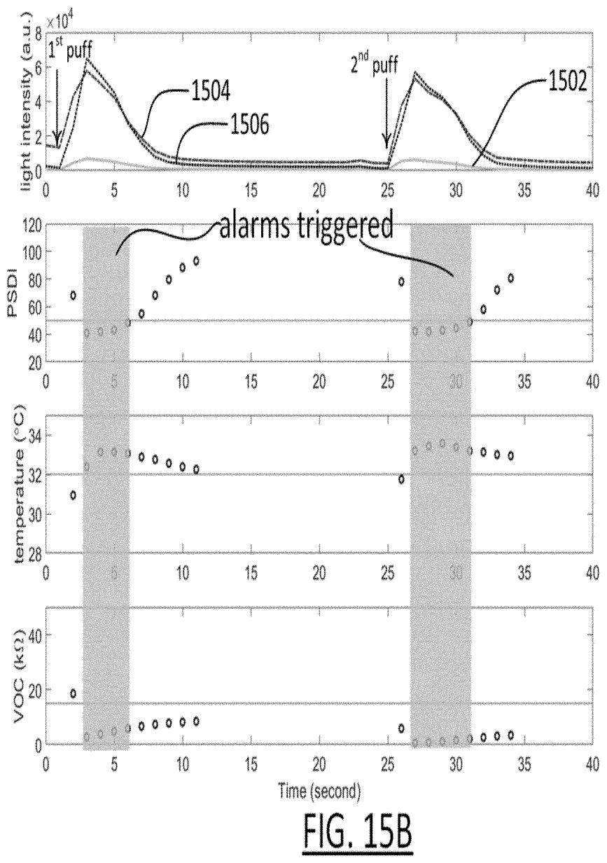

FIG. 15A and FIG. 15B show experimental results collected from an example electronic cigarette device 200 including the aerosol particle senor 60 (using the LED assembly 652 and the photodetector 654, based on photometric measurement), the temperature sensor 70, and the chemical sensor 80 (using the metal oxide sensing layer 852 to measure volatile organic compounds). The curves 1506, 1504, and 1502 were the measured scattered light intensity I_{λ1}, I_{λ2}, and I_{λ3} for wavelength 880 nm (λ₁), 660 nm (λ₂), and 527 nm (λ₃), respectively. The PSDI was calculated using equation (2). The safety threshold for PSDI is 50. The safety threshold for aerosol temperature is 32° C. The safety threshold for electrical resistance of the chemical sensor is 15 kΩ. Results in FIG. 15A were collected from the device operating with a good condition of 30 Watts power on the atomizer. The results show the generated aerosol was in good quality and no warning was triggered. Results in FIG. 15B were collected from the device operating with an overheated atomizer at 60 Watts power. The PSDI was below the safety threshold indicating too many larger particles in the aerosol, the aerosol temperature was above the safety threshold, and the concentration of volatile organic compounds was too high (electrical resistance below the safety threshold). The alarms were triggered in every puff to warn the user about the harmful aerosol.

The above-described methods 1000 and 1100 can be used in conjunction with the electronic cigarette device 200 as described herein. These two methods can also be used in conjunction with the inhalation drug delivery devices 400 of the invention, including without limitation, a medical nebulizer, a pressurized metered-dose inhaler, and a dry powder inhaler. In the case of medical nebulizer, the aerosol generator can include a compressor, tubing, and nebulizer cup, and the drug solution can be loaded inside the nebulizer cup. When a low-quality aerosol is detected, the user can be warned immediately in order to prevent the user from inhaling the generated aerosol. The measured parameters can be compared with desired parameters to evaluate the differences between the generated aerosol and the ideal aerosol. Additional settings can also be used to modify the functionality of the aerosol generator to improve the quality of the generated aerosol to reach the desired characteristics.

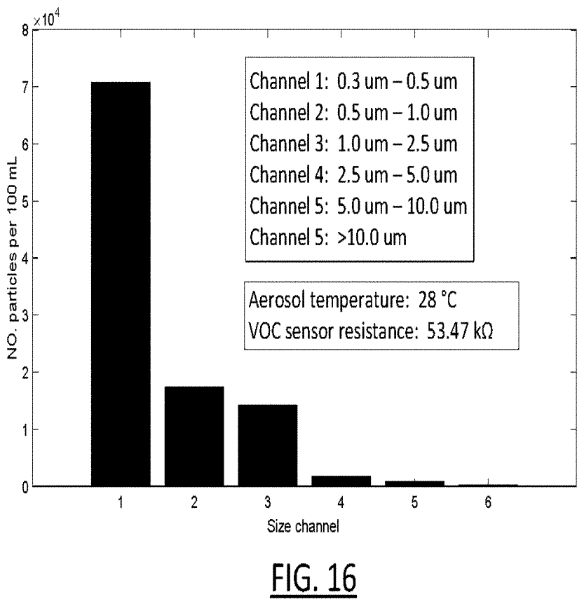

FIG. 16 shows experimental results collected from an example medical nebulizer 400 including the aerosol particle senor 60 (using the laser diode 602 and the photodetector 606, based on optical particle counting), the temperature sensor 70, and the chemical sensor 80 (using the metal oxide sensing layer 852 to measure volatile organic compounds (VOC)). The chart shows the number of particles per 100 mL in each specified aerosol particle channel, which were calculated based on the principle of optical particle counting. The measured aerosol temperature was 28° C. and the measured electrical resistance from the chemical sensor was 53.47 kΩ. It can be appreciated that, as disclosed herein, the quality of the generated aerosol can be controlled and maintained for achieving optimal user experiences while minimizing health risks for the user at the same time using the system of the present disclosure. The efficacy of the drug delivery through the aerosol can also be controlled accurately for achieving desired efficacy.

Certain variations can be made to the above-described methods, including without limitation, adding steps of signal processing, adding machine learning algorithm to decide thresholds for detecting harmful aerosols, pairing the aerosol delivery device 100 with a mobile device via Bluetooth communication, combinations and sub-combinations of any of the above, without deviating from the spirit of the present disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An electronic aerosol safety system to warn a user of a target in an aerosol stream, comprising:
at least one aerosol safety sensor for measuring or sensing one or more parameters of the target in the aerosol stream; and
warning means in communication with the sensor, the warning means for providing a user target parameter deviation notification.

2. The electronic aerosol safety system of claim 1, wherein the at least one aerosol safety sensor comprises at least one sensor selected from a group consisting of an aerosol particle sensor, a temperature sensor, a chemical sensor, and combinations thereof.

3. The electronic aerosol safety system of claim 2, wherein the chemical sensor includes a sensor selected from the group consisting of an electrochemical sensor, a metal oxide sensor and combinations thereof.

4. The electronic aerosol safety system of claim 2, wherein the aerosol particle sensor includes a sensor selected from a group consisting of an optical particle counting sensor, a dynamic light scattering sensor, a photometric particle measuring sensor, and combinations thereof.

5. The electronic aerosol safety system of claim 4, wherein the optical particle counting sensor includes an optical configuration selected from a group consisting of a regular perpendicular configuration and a reflective configuration.

6. The electronic aerosol safety system of claim 4, wherein the dynamic light scattering sensor includes an optical configuration selected from a group consisting of a regular perpendicular configuration and a reflective configuration.

7. The electronic aerosol safety system of claim 4, wherein the photometric measuring sensor includes at least one light emitting diode and at least one photodetector.

8. The electronic aerosol safety system of claim 1, wherein the warning means includes an information display for displaying target parameters present in the aerosol stream.

9. The electronic aerosol safety system of claim 8, wherein the warning means includes a visual notification for alerting the user to the target parameter deviation notification.

10. The electronic aerosol safety system of claim 9, wherein the warning means includes a member selected from a group consisting of a light, a vibration, and combinations thereof.

11. The electronic aerosol safety system of claim 1, wherein the electronic aerosol safety system is integrated into an aerosol delivery system.

12. The electronic aerosol safety system of claim 1, wherein the electronic aerosol safety system includes attachment means for attaching the electronic aerosol safety system to an aerosol delivery system.

13. The electronic aerosol safety system of claim 1, wherein the electronic aerosol safety system is integrated into an aerosol delivery system selected from a group consisting of an electronic cigarette device and an inhalation drug delivery device.

14. A method of increasing the safety of an aerosol delivery device for a user, comprising:
connecting an electronic aerosol safety system according to claim 1 to a body of the aerosol delivery device;
monitoring the aerosol produced by the aerosol delivery device by detecting at least one preset parameter using the electronic aerosol safety system; and
triggering a notification to the user of the aerosol delivery device upon detection of a deviation from the preset parameter.

15. The method of claim 14, wherein the triggering step includes vibrating the aerosol delivery device using a vibration motor.

16. The method of claim 14, wherein the triggering step includes displaying notification information on a display screen.

17. The method of claim 14, wherein the triggering step includes pushing notification information to the user on a connected mobile device.

18. The method of claim 14, further comprising controlling the aerosol delivery system by modulating the activity of the aerosol delivery device to generate an aerosol within at least one preset parameters.

19. A method of increasing the efficacy of an aerosol delivery device by:
connecting an aerosol safety system according to claim 1 to a body of the aerosol delivery device;
measuring parameters of the aerosol produced by the aerosol delivery device by detecting preset parameters using the aerosol safety system; and
evaluating the difference between the measured parameters of the aerosol and the desired parameters of the aerosol; and
adjusting aerosolization conditions on the aerosol generator to reach target parameters.

20. A remote system for communicating with the aerosol safety system of claim 1, the remote system comprising:
wireless communication devices for wirelessly receiving information transmitted from the aerosol safety system;
software connected to the wireless communication devices for receiving, analyzing, and tracking the information, and for configuring the desired parameters of the aerosol; and
a transmitter for transmitting control instruction from the software to the aerosol safety system and aerosol delivery device.
